(19) United States
(12) Patent Application Publication
(10) Pub. No.: US 2017/0242018 A1
(43) Pub. Date: Aug. 24, 2017

(54) Methods, Systems, and Compositions for Detection of Aldehydes

(71) Applicant: Pulse Health, LLC, Lake Oswego, OR (US)

(72) Inventors: Gerald Thomas, Springfield, OR (US); Juven Lara, Portland, OR (US); Charles Noll, Portland, OR (US); Brian Young, Portland, OR (US); Craig Carlsen, Lake Oswego, OR (US); Maura Mahon, Lake Oswego, OR (US); James Ingle, Corvallis, OR (US)

(21) Appl. No.: 15/436,103

(22) Filed: Feb. 17, 2017

Related U.S. Application Data
(60) Provisional application No. 62/296,947, filed on Feb. 18, 2016.

(51) Int. Cl. G01N 33/58; G01N 33/64

(52) U.S. Cl. CPC G01N 33/582; G01N 33/64

(57) ABSTRACT

Methods, systems and reagents are provided for detecting and quantifying carbonyl containing moieties in a variety of sample types. The amount of time elapsed from capturing of the carbonyl containing moieties from a sample to the detection of the carbonyl containing moieties is less than about 2 hours. Compounds are provided to facilitate labeling and detection of the carbonyl containing moieties.

### CROSS-REFERENCE TO RELATED APPLICATION

This application claims the benefit under 35 U.S.C. §119(e) of U.S. Provisional Patent Application No. 62/296,947, filed Feb. 18, 2016, and entitled “Breath Analysis System,” the contents of which are incorporated by reference as if fully disclosed herein.

### TECHNICAL FIELD

The present disclosure is directed to the field of carbonyl detection and quantitation, and in particular the detection and quantitation of carbonyl containing moieties in biological samples.

### BACKGROUND

Oxidative stress is indicative of an imbalance between the production of reactive oxygen species and the ability of the body to detoxify the reactive compounds. Oxidative stress is commonly defined as a pathophysiologic imbalance between oxidative and reductive (anti-oxidative) processes (or oxidants>antioxidants). When the imbalance exceeds cellular repair mechanisms oxidative damage accumulates. Elevated levels of reactive oxidant species are associated with the pathogenesis of a variety of diseases from cardiovascular, pulmonary, autoimmunological, neurological, inflammatory, connective tissues diseases, and cancer. Oxidative stress results in tissue damage and is reportedly involved in diabetes mellitus, hearing loss, vascular disease, neural disease, kidney disease, and much more. Dietary consummation of antioxidants is recommended to combat and prevent a number of diseases and is associated with general health and well-being.

Measuring oxidative stress levels in an individual or patient population can be desirable, but attempts to identify and measure molecules associated with oxidative stress are typically associated with invasive techniques including blood draws, urine samples, and tissue samples. In addition, reactive oxygen molecules associated with oxidative stress are extremely reactive and have short half-lives within and outside the body making direct measurement extremely difficult and inaccurate. At this point a convenient and easy measure of oxidative stress status is not available.

Given the absence of effective methods and devices for identifying individuals or patient populations with oxidative stress, there is a need to advance the industry to better human health.

### SUMMARY

Provided herein are methods for detecting the presence of at least one carbonyl containing moiety in a sample. The method comprises the steps of: exposing the sample to a substrate to capture the carbonyl containing moiety; eluting the carbonyl containing moiety off the substrate; mixing the carbonyl containing moiety with a reactive labeling agent; injecting the labeled carbonyl containing moiety onto a column; eluting the labeled carbonyl containing moiety from the column in an organic solvent; and detecting the labeled carbonyl containing moiety. In some aspects, the method of detecting is complete in less than about 2 hours. In some embodiments, the method further comprises measuring the concentration of the at least one carbonyl containing moiety.

Provided herein are methods for detecting the presence of at least one aldehyde in a sample. The methods comprise the steps of: exposing the sample to a substrate to capture the aldehyde; eluting the aldehyde off the substrate; mixing the aldehyde with a reactive labeling agent; injecting the labeled aldehyde onto a column; eluting the labeled aldehyde from the column in an organic solvent; and detecting the labeled aldehyde. In some aspects, the method of detecting is complete in less than about 2 hours. In some embodiments, the method further comprises measuring the concentration of the at least one aldehyde.

Provided herein are methods of detecting carbonyl containing moieties in a gas sample. The methods comprise: isolating carbonyl containing moieties from a sample; mixing the carbonyl containing moieties with a reactive labeling agent, wherein the carbonyl containing moieties associate with the reactive labeling agent; passing the labeled carbonyl containing moieties through a column; exciting the labeled carbonyl containing moieties exiting the column; and detecting the carbonyl containing moieties by measuring the fluorescence emitted from or absorbed by the reactive labeling agent associated with the carbonyl containing moieties. In some aspects, the step of eluting resolves the carbonyl containing moieties based on the carbon chain length. In some aspects, the time elapsed from isolating the carbonyl containing moieties from the sample to detecting the carbonyl containing moieties is less than about 2 hours.

Provided herein are compounds comprising a fluorophore, a linker, and a reactive group. In some embodiments, the fluorophore is selected from the group consisting of ao-5-TAMRA, ao-6-TAMRA, and mixtures thereof. In some embodiments, the linker is selected from the group consisting of hexanoic acid, aminohexanoic acid, cadavarine, polyethylene glycol, and polyglycol. In some embodiments, the reactive group is selected from the group consisting of a hydrazine moiety, a carbohydrazide moiety, a hydroxylamine moiety, a semi-carbazide moiety, an aminooxy moiety, and a hydrazide moiety.

Provided herein are systems for detecting the presence of at least one carbonyl containing moiety in a sample. The systems comprise: a substrate to capture the carbonyl containing moiety; reagents for eluting the carbonyl containing moiety off the substrate; reagents for associating the carbonyl containing moiety with a reactive labeling agent; a column for resolving the labeled carbonyl containing moiety; solvents for eluting the labeled carbonyl containing moiety from the column; and a light and detector for generating fluorescence excitation, absorbance, and/or emission to detect the labeled carbonyl containing moiety. In some aspects, the system completes one cycle in less than about 2 hours. In some embodiments, the system further comprises standards for measuring the concentration of the at least one carbonyl containing moiety.

### BRIEF DESCRIPTION OF THE DRAWINGS

FIG. 1 illustrates a system in which target molecules are labeled with a selective reactive fluorophore and separated to allow identification of individual aldehydes differing by 1 carbon in chain length.

FIG. 2 demonstrates an illustrative reactive labeling agent comprising a dye, a linker, and a reactive group.

FIG. 3 provides the structures of ao-5-TAMRA and ao-6-TAMRA, modified according to the methods provided herein to generate reactive labeling agents comprising a linker and a reactive group attached to the fluorophores.

FIG. 4 provides a synthesis schematic of a reactive labeling agent comprising ao-6-TAMRA.

FIG. 5 illustrates the benefits of using a linker, for example, PEG, in a reactive labeling agent.

FIG. 6 illustrates the reaction rate as a function of pH, in the absence of a catalyst, at room temperature.

FIG. 7 illustrates the effect of use of catalysts, 5-MAA or 3,5-DABA, on reaction rate.

FIG. 8 shows a fluorescence chromatograph of a serial dilution of a mixture of ao-6-TAMRA-labeled aldehydes along with reactive and non-reactive internal standards.

FIG. 9 provides a SPE separation analysis demonstrating isolation of aldehydes by group. FIG. 9 further demonstrates the use of varying organic solvents or concentrations thereof to separate closely related molecules.

FIG. 10 compares two chromatographs in which the aldehydes were separated on 10 μm semi-prep guard columns of two different lengths, 30 mm and 50 mm.

FIG. 11 compares two chromatographs, the upper based on a sample containing reference C3-C10 aldehydes and the lower based on a breath sample. Upper: A sample containing the products formed with the flurorphore and C3-C10 aldehyde was used as a reference. Lower: A breath sample compared to the standard to verify assignment of the products. Labeling: 6.8 μM 6-ao-TAMRA, 3 mM 5-MAA, 70 mM citrate, pH 4.2, 40% MeOH. Collection 10 L TEDLAR bag, Capture 300 mg CUCIL silica, Elution 1.26 mL, 40% MeOH. Incubation 15 min at room temperature. Separation: 4.6 mm×50 mm, 10 μm C18 phenomenex, 45-100% MeOH. Detection: Agilent 1100 Fl detector G1321.

FIG. 12 shows two chromatographs obtained using devices with different designs. Device Detector 1: 90 degree geometry, 532 nm excitation, 20 mw laser, flow cell: 1 mm ID Tefzel plastic tube, 2 mm mask (slit), collection 25.4 mm cylindrical lens, LP filter semrock 561 nm, fiber 600 μm core, detector USB-2000 CCD (ocean optics) band pass 560-610 nm, 100 msec integration, box car 5, scans 20, 50 femtomoles of C6. Device Detector 2: 90 degree geometry, 532 excitation, 20 mw laser, cell 500 μm capillary (Polymicro TSP500794) 15 mm focus lens, beam splitter, 16 mm collection lens, LP filer omega 550 nm. detector USB-2000 CCD (ocean optics) band pass 560-610 nm, 100 msec integration, box car 5, scans 20. 1 femtomole each of aldehyde C4-C10 labeled with ao-6-TAMRA.

FIG. 13 demonstrates the reactivity of the labeling agent.

FIG. 14 shows a chromatograph of aldehydes labeled with a reactive labeling agent comprising mixed isomers of ao-5,6-TAMRA.

FIG. 15 compares two chromatographs of aldehydes labeled with reactive labeling agents comprising either ao-5-TAMRA or ao-6-TAMRA.

FIG. 16 shows efficiency of labeling as a function of temperature and time.

FIG. 17 demonstrates the effect of a catalyst on reaction rates. Without a catalyst and at low analyte concentrations, the labeling reaction is slow. The reaction rate can increase 10 fold in the presence of a catalyst. The reaction conditions were 1:1.2 reactive labeling agent (comprising ao-5,6-TAMRA):hexanal, 5-MMA at molar ratio of 1, 100, and 1000; 6.5 mM citrate, pH 4.16, room temperature.

FIG. 18 provided limits of detection (LOD) curve using serial dilutions of aldehydes in equal concentrations. Reactive (C12 aldehyde) and non-reactive (C16 amide) internal standards were added at constant concentrations to each sample in the dilution series. The reaction was incubated for 15 minutes then quenched. Mixtures were analyzed by HPLC under standard conditions, 4×20 mm, 5 μm, C18 column.

FIG. 19 provides chromatographs comparing a breath sample to a standard sample where the reactive labeling agent comprises ao-6-TAMRA. Using peak height, the estimate for C3-C10 as a sum is approximately 80 pmole/L or 2.2 ppb. The sum for C4-C10 is estimated at 48 pmole/L or 1.2 ppb. Labeling: 6.8 μM 6-ao-TAMRA, 3 mM 5-MAA, 70 mM citrate pH 4.2 40% MeOH, Collection 10 L TEDLAR bag, Capture 300 mg CUCIL silica, Elution 1.26 mL 40% MeOH. Incubation 15 min at room temperature. Separation: 4.6 mm×50 mm, 10 μm C18 phenomenex. 45-100% MeOH. Detection: device detector 1 (see FIG. 12).

FIG. 20 demonstrates the labeling reaction as a function of the concentration of the reactive labeling agent comprising ao-6-TAMRA. The reactive labeling agent concentration varied from 0.5 μM to 20 μM. Maximum signal was observed at approximately 10 μM.

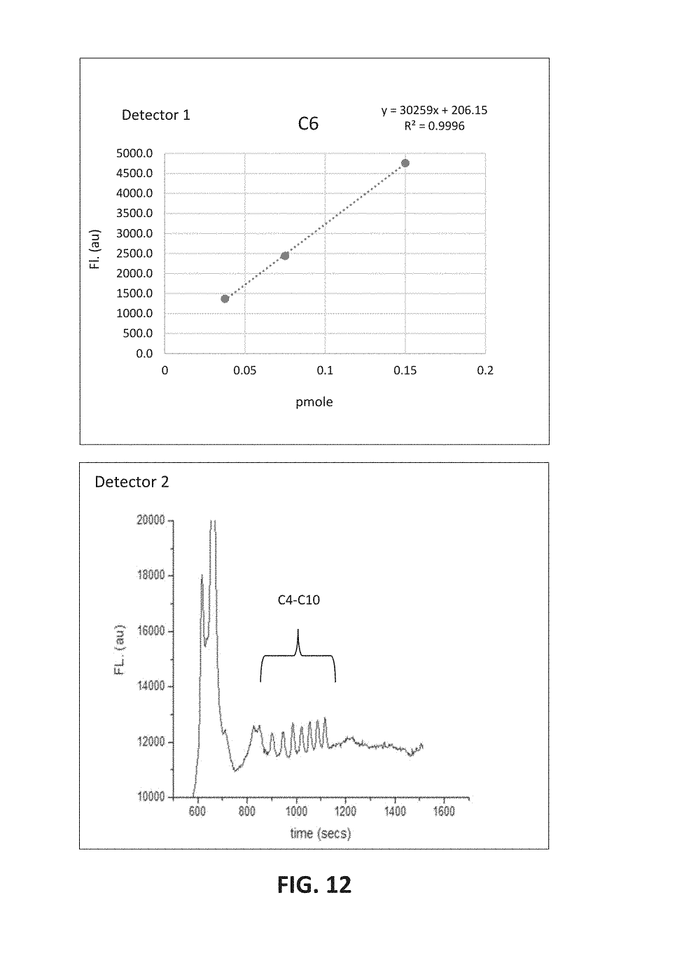

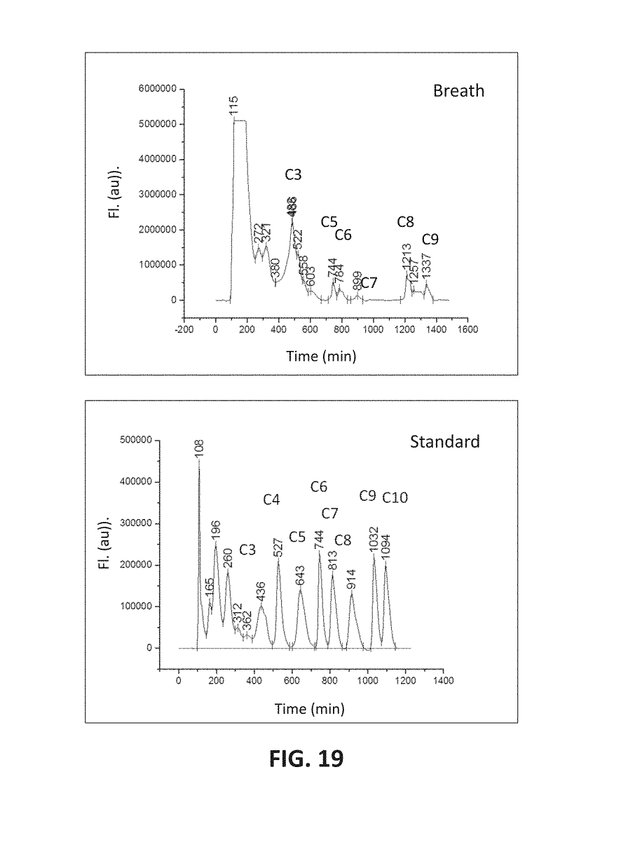

With the exception of FIGS. 12 and 19, all chromatographic data were acquired using Agilent (Hewitt-Packard) Model 1100 HPLC systems equipped with diode array and fluorescence detection. For TAMRA, excitation 550 nm (with a band width of 20 nm, i.e 540-560 nm) and emission 580 nm (with a band width of 20 nm, i.e. 570-590 nm). Typical separation method, mobile phase methanol, aqueous 10 mM TEAA pH 7. Linear gradient 45% to 100% methanol, flow rate 1 ml/min.

### DETAILED DESCRIPTION

The description, experiments, and drawings provided herein are illustrative and are not to be construed as limiting. Numerous specific details are described to provide a thorough understanding of the disclosure. However, in certain instances, well-known or conventional details are not described in order to avoid obscuring the description. References to one or another embodiment in the present disclosure can be, but not necessarily are, references to the same embodiment; and, such references mean at least one of the embodiments.

Reference in this specification to “one embodiment” or “an embodiment” means that a particular feature, structure, or characteristic described in connection with the embodiment is included in at least one embodiment of the disclosure. Appearances of the phrase “in one embodiment” in various places in the specification do not necessarily refer to the same embodiment, nor are separate or alternative embodiments mutually exclusive of other embodiments. Moreover, various features are described which may be exhibited by some embodiments and not by others. Similarly, various requirements are described which may be requirements for some embodiments but not other embodiments.

The terms used in this specification generally have their ordinary meanings in the art, within the context of the disclosure, and in the specific context where each term is used. Certain terms that are used to describe the disclosure are discussed below, or elsewhere in the specification, to provide additional guidance to the practitioner regarding the description of the disclosure. For convenience, certain terms may be highlighted, for example using italics and/or quotation marks: The use of highlighting has no influence on the scope and meaning of a term; the scope and meaning of a term is the same, in the same context, whether or not it is highlighted. It will be appreciated that the same thing can be said in more than one way.

Consequently, alternative language and synonyms may be used for any one or more of the terms discussed herein. Nor is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and is not intended to further limit the scope and meaning of the disclosure or of any exemplified term. Likewise, the disclosure is not limited to various embodiments given in this specification.

Without intent to further limit the scope of the disclosure, examples of methods, systems, reagents, and compounds according to the embodiments of the present disclosure are given below. Note that titles or subtitles may be used in the examples for convenience of a reader, which in no way should limit the scope of the disclosure. Unless otherwise defined, all technical and scientific terms used herein have the same meaning as commonly understood by one of ordinary skill in the art to which this disclosure pertains. In the case of conflict, the present document, including definitions, will control.

Provided herein are methods, systems, reagents, and compounds useful for the detection, quantitation and assay of carbonyl containing moieties (“CCM”) including aldehydes, ketones, and carboxylic acids. A CCM is a compound having at least one carbonyl group. A carbonyl group is the divalent group >C=0, which occurs in a wide range of chemical compounds. The group consists of a carbon atom double bonded to an oxygen atom. The carbonyl functionality is seen most frequently in three major classes of organic compounds: aldehydes, ketones, and carboxylic acids. It is contemplated herein that the disclosed methods, reagents, and systems are useful in resolving, detecting, and quantitating mixtures of CCMs.

The methods, reagents, compounds, and systems provided herein are useful in detecting the presence and/or concentration of aldehydes in a variety of samples. Exemplary aldehydes include without limitation 1-hexanal, malondialdehyde, 4-hydroxynonenal, acetaldehyde, 1-propanal, 2-methylpropanal, 2,2-dimethylpropanal, 1-butanal, and 1-pentanal. Exemplary aldehydes include C1 aldehydes, C2 aldehydes, C3 aldehydes, C4 aldehydes, C5 aldehydes, C6 aldehydes, C7 aldehydes, C8 aldehydes, C9 aldehydes, C10 aldehydes, C11 aldehydes, C12 aldehydes, and C13 aldehydes. Exemplary aldehydes include aliphatic aldehydes, di-aldehydes, and aromatic aldehydes. It is contemplated herein that the disclosed methods, reagents, and systems are useful in resolving, detecting, and quantitating mixtures of aldehydes. In some embodiments, the sample comprises two or more aldehydes of different carbon chain lengths, and the step of eluting the labeled aldehyde resolves each aldehyde based on carbon chain length.

The methods, reagents, compounds, and systems provided herein have a wide range of utility in a variety of applications in which indication of the presence and/or estimation of concentration of a CCM, such as an aldehyde, a ketone, or a carboxylic acid, is useful.

As used herein, the term “an aldehyde” is intended to refer to any compound that may be chemically characterized as containing one or more aldehyde functional groups. In some embodiments, a pass/fail type indication will be made indicating that some minimum concentration of a specific aldehyde or group of aldehydes is present. In some embodiments, an estimation of the concentration is made. Various embodiments are designed to be specific for specific aldehyde(s), for groups of aldehydes of interest, or for all aldehydes in a sample.

Illustratively, methods and systems provided herein can specifically measure the presence and/or concentration of malondialdehyde, an unsaturated molecule with two aldehyde functional groups, from biologic samples (breath, urine, blood, saliva, others) or environmental samples (water, air, etc.). Detection of aldehydes in a biologic sample can be useful for indicating oxidative stress in living beings. In some embodiments, methods, reagents, compounds, and systems provided herein are useful to measure other various compounds containing one or more aldehyde groups, including saturated and/or unsaturated molecules, as biomarkers for various diseases and conditions. The aldehyde concentration in human breath can serve as a biomarker useful to screen for the presence of lung cancer.

Other embodiments include applications useful in food and agricultural related testing. The oxidation of oils has important effects on the quality of oily foods. Such oxidation generates aldehydes, including the unsaturated aldehydes 2-heptenal, 2-octenal, 2-decenal, 2-undecenal and 2,4-decadienal, and/or trans molecules of these compounds. Similarly, levels of formaldehyde and acetaldehyde in fish and seafood can indicate quality. Lipids present in foods react with oxygen and other substances to produce aldehydes, and the level of lipid oxidation (and hence the concentration of aldehydes) can be indicative of food quality. Other applications include environmental and others in which aldehyde presence in gasses or liquids can be indicative of gas or liquid quality or pollution thereof.

Aldehydes can be detected and/or quantitated in order to provide information on the general health and wellness of a subject, for example, a patient. In some embodiments, the information can be indicative of a patient's level of oxidative stress. In some embodiments, aldehydes may be measured or analyzed to assist in the medical diagnosis of a patient. For example, aldehydes in breath (or urine, blood, plasma, or headspace of cultured biopsied cells) may be sampled to determine a patient's overall health and/or whether the patient suffers from certain medical conditions. Aldehyde sampling may indicate whether a patient has cancer, for example, esophageal and/or gastric adenocarcinoma, lung cancer, colorectal cancer, liver cancer, head cancer, neck cancer, bladder cancer, or pancreatic cancer, may indicate whether a patient suffers from a pulmonary disease (including asthma, acute respiratory distress syndrome, tuberculosis, COPD/emphysema, cystic fibrosis, and the like), neurodegenerative diseases, cardiovascular diseases, or is at risk of an acute cardiovascular event, infectious diseases (including mycobacterium tuberculosis, pseudomonas aeruginosa, aspergillus fumigatus, and so on), gastrointestinal infections (including Campylobacter jejuni, Clostridium difficile, H. pylori, and the like), urinary tract infections, sinusitis, and other conditions. Aldehyde sampling may also indicate the severity or staging of a particular disease or condition.

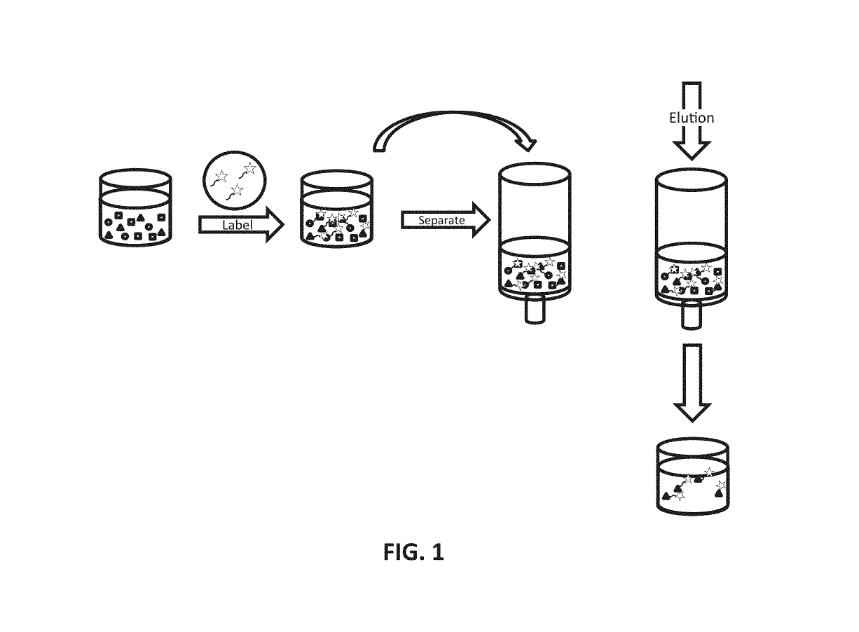

Provided herein are reagents, compounds, systems, and methods for detecting and quantitating CCM, including aldehydes, ketones, and carboxylic acids. Illustratively, the detection and quantitation of alkyl aldehydes, by-products of lipid peroxidation associated with oxidative stress and oxidative biological processes, can inform a care-giver or practitioner regarding the oxidative stress status of a subject. Interesting attributes of the disclosure include selective reactive “painting” of the desired targets, e.g. CCM such as aldehydes, and specific isolation and detection of the labeled target (See FIG. 1).

In accordance with one embodiment, there is provided a method and system that includes exposing a sample to a substrate to capture the aldehyde; eluting the aldehyde off the substrate; mixing the aldehyde with a reactive labeling agent; isolating, detecting, and optionally quantitating the desired labeled aldehydes. The process is sufficiently rapid to provide for on-site measurements and reporting of results. For example, in some embodiments, the process from capture of the aldehydes to detecting of the aldehydes can be completed in less than about 2 hours, or less than about 1.5 hours, or less than about 75 minutes, or less than about 1 hour.

### Sample Sources

As used herein, a “biological sample” is referred to in its broadest sense, and includes solid, gas, and liquid or any biological sample obtained from nature, including an individual, body fluid, cell line, tissue culture, or any other source. As indicated, biological samples include body fluids or gases, such as breath, blood, semen, lymph, sera, plasma, urine, synovial fluid, spinal fluid, sputum, pus, sweat, as well as gas or liquid samples from the environment such as plant extracts, pond water and so on. Solid samples may include animal or plant body parts, including but not limited to hair, fingernail, leaves and so on. The biological sample for one embodiment provided herein is the breath of a human.

Though the methods, reagents, compounds, and systems provided herein can apply to a variety of sample types, in the medical use context, breath analysis represents a promising non-invasive alternative to serum chemistry. A compendium of volatile organic compounds (VOCs) with relatively low molecular weight reflects distinct and immediate changes as a result of alterations in pathophysiological processing and metabolism. Changes in the appearance and population of VOCs in breath reflect changes in metabolism and disease states. Provided herein are methods and systems for detection and differentiation of diseases from exhaled breath.

### Illustrative Methods and Systems

Provided herein is a non-invasive system for the quantification of oxidative stress status. Oxidative stress is commonly defined as a pathophysiologic imbalance between oxidative and reductive (anti-oxidative) processes (or oxidants>antioxidants). When the imbalance exceeds cellular repair mechanisms, oxidative damage accumulates. Elevated levels of reactive oxidant species are associated with the pathogenesis of a variety of diseases from cardiovascular, pulmonary, autoimmunological, neurological, inflammatory, connective tissues diseases and cancer. However, by-products of lipid oxidation in breath and other biological samples are present in such low quantities exceeding the limit of detection of conventional devices and methods. Furthermore, these same by-products are not stable in a sample over time, and attempts to identify or quantitate such molecules are unsuccessful due to degradation prior to or during analysis.

Provided herein are methods, reagents, and systems for measuring oxidative stress. In some embodiments, the methods and systems detect and/or quantitate by-products of lipid oxidation, for example, alkyl aldehydes and ketones. In some embodiments, these by-products are measured in a sample of exhaled breath. The methods comprise selective reactive “painting” of the chemical class of desired targets and specific isolation and detection of the “desired” subclass of “painted” or labeled targets.

In some embodiments, there are provided methods for identifying and/or measuring an aldehyde in a sample, the methods comprise providing a device for capturing a biological sample, where the device includes a substrate for capturing aldehydes, includes a reactive labeling agent for labeling aldehydes, includes a column for separating classes of aldehydes, includes a light for inducing fluorescence, and includes a detector for measuring fluorescence emission, excitation, or absorbance.

In some embodiments, the device receives a breath sample containing aldehydes from a subject, deposits the sample on a substrate, performs an elution process on the sample to capture the aldehydes, mixes and incubates the aldehydes with a reactive labeling agent, separates and measures the labeled aldehydes, and presents measurement results.

In some embodiments, there are provided methods for identifying and/or measuring a CCM such as an aldehyde, a ketone, or carboxylic acid. In some embodiments, the reactive labeling agent attaches to the aldehydes present in a sample and the remaining components in the sample are removed as is the unbound reactive labeling agent. In some embodiments, a reverse phase matrix or stacked matrices can be used to separate labeled aldehydes for measuring.

In some aspects, the method can include capturing aldehydes from a biological sample on a substrate, eluting the aldehydes from the substrate, and labeling the aldehydes. In some aspects, the method can include capturing aldehydes from a biological sample on a substrate, labeling the captured aldehydes, and eluting the labeled aldehydes. In some aspects, the substrate is incorporated with the reactive labeling agent.

In some embodiments, the device comprises a fluorescence detection assembly that includes an emitter, a detector, a light chamber, a fluorescence chamber and a well, a light path that extends from the emitter, through the light chamber and through the well, and a fluorescence path that extends from the well, through the fluorescence chamber and to the detector.

In some embodiments, a method of detecting fluorescence includes exciting a solution containing fluorescently labeled carbonyl containing moieties. The light passes through the solution and excites the fluorescently labeled moieties producing a fluorescence, and the fluorescence absorbance or emission is detected.

In some embodiments, a method for detecting and quantifying carbonyl containing moieties in breath includes (a) obtaining a biological sample, (b) capturing carbonyl containing moieties from the sample on a substrate, (c) labeling the carbonyl containing moieties to provide a labeled solution, (d) directing light within a predetermined wavelength range through the labeled solution, thereby producing a fluorescence, and (e) detecting the fluorescence.

In some embodiments, the labeling step (c) comprises mixing (i) the CCM with (ii) the buffer, and then adding (iii) the catalyst and lastly (iv) the reactive labeling agent. In some embodiments, (ii) the buffer can be present in the elution solution, such that (ii) the buffer is present in solution with (i) the carbonyl containing moiety. In some embodiments, internal standards are added to the solution prior to the addition of the catalyst. Addition of the catalyst and the reactive labeling agent last can help prevent pre-incubation and loss of reactivity.

As such, provided herein are compositions comprising CCM such as aldehydes captured from a sample, a buffer, and a catalyst. In some embodiments, the compositions further comprise a reactive labeling agent. In some embodiments, the compositions further comprise at least one non-reactive internal standard. In some embodiments, the compositions further comprise at least one reactive internal standard. In some embodiments, the composition consists essentially of CCM such as aldehydes captured from a sample, a buffer, a catalyst, a reactive labeling agent, and optionally at least one internal standard.

It will be appreciated that any biological sample can be analyzed using the system. Breath constituents other than CCM or aldehydes can be captured and analyzed as desired. U.S. Patent Publication Nos. 2003/0208133 and 2011/0003395 are incorporated by reference herein in their entireties.

### Target Capture

The system and methods provided herein are amenable to “real-time” assay formats for the detection of CCM, and can be applied to the detection of CCM in solution, and/or the detection of trace CCM in the gas phase by the addition of a primary capture (on a substrate) and release (elution from the loaded substrate) process. In one step of the process, gas phase CCM, for example, aldehydes from the breath of a human, are captured on a substrate.

The capture substrate contemplated as useful herein is desirably formed from a solid, but not necessarily rigid, material. The solid substrate may be formed from any of a variety material, such as a film, paper, nonwoven web, knitted fabric, woven fabric, foam, glass, etc. For example, the materials used to form the solid substrate may include, but are not limited to, natural, synthetic, or naturally occurring materials that are synthetically modified, such as polysaccharides (e.g., cellulose materials such as paper and cellulose derivatives, such as cellulose acetate and nitrocellulose); polyether sulfone; polyethylene; nylon; polyvinylidene fluoride (PVDF); polyester; polypropylene; silica; inorganic materials, such as deactivated alumina, diatomaceous earth, MgSO₄, or other inorganic finely divided material uniformly dispersed in a porous matrix, with polymers such as vinyl chloride, vinyl chloridepropylene copolymer, and vinyl chloride-vinyl acetate copolymer; cloth, both naturally occurring (e.g., cotton) and synthetic (e.g., nylon or rayon); porous gels, such as silica gel, agarose, dextran, and gelatin; polymeric films, such as polyacrylamide; and so forth. In some aspects, the substrate is a solid phase matrix of silica optionally spaced between frits. The size of the substrate is chosen so that a measurable amount of CCM is captured by the substrate. The size can vary but generally it is about 2 mL, or about 1 mL, or about 0.25 mL.

The substrate typically consists of a bed of particles with 50-60 angstrom pores, with a 50-270 mesh (300-50 μm), and a mass of 75 to 300 mg, or 60-120 mesh (250-125 μm) and a mass of 100 to 200 mg, or 50-120 mesh (210-125 μm) and a mass of 125 to 300 mg, or 200-325 mesh (80-44 μm) with a mass of 75 to 500 mg.

The amount of a CCM captured by the substrate may vary, but typically for a substrate consisting of 200 mg of 50-270 mesh (300-500 μm) particle with a bed diameter of 12.5 mm, generally, it will be equivalent to the amount in a human's breath after breathing into a tube like a breathalyzer. In some aspects, it will be from 75 to 0.1 ppb (400 to 4 pmoles), or from 20 ppb to 0.01 ppb (80 to 0.4 pmoles).

In general, the elution solution of the captured aldehyde from the capture matrix includes a buffer and/or an organic solvent. The organic solvent can include methanol, ethanol, propanol, isopropanol, and/or acetonitrile, and can be present in an amount of about 34% to 50%, or about 35%, about 38%, about 40%, about 45%, etc. The concentration of the buffer can range from 10 mM to 100 mM. In some embodiments, a surfactant is substituted for the solvent.

A salt can optionally be included and can be any salt that does not negatively impact the fluorescing solution and controls salting effects in the elution solution. Salts contemplated herein can include NaCl, LiCl, KCl, sulfates and phosphates, and mixtures thereof. The concentration of the salt can range from 5 mM to 100 mM.

The buffer is employed to maintain the elution solution mildly acidic and at a pH of between 2 and 6, or about 2.5, or about 4, or about 4.2. The buffer can be HCl, a borate buffer, a phosphate buffer, a citrate buffer, acetic acid/acetate & citrate/phosphate.

The temperature for practicing the methods provided herein can range from 15 to 35° C., for example, from 25 to 30° C.

### Label and Separate Process and Systems

In this process, the targets, aldehydes and ketones, are labeled with carbonyl selective reactive fluorescent “paint”.

The label serves two purposes: 1) transform the “transparent” alkyl aldehyde targets into a species that can be observed and quantitated by either absorption or fluorescence emission detection and 2) enable and enhance the selective isolation of the desired targets.

The label and separation matrix provides a combination of reactivity, signal, and separation properties useful in the embodiments provided herein, and provides the ability to resolve and identify individual aldehydes that differ by a single carbon in chain length.

In some embodiments, classes of labeled aldehydes can be isolated into “bulk” classes using low resolution 60-200 μm particles normally found in SPE columns. In this embodiment, groups of similar chain length aldehydes, i.e. C1-C3, C5-C10, can be isolated and detected in bulk providing for rapid analysis of groups of selected aldehydes.

The labeled aldehydes can be isolated in bulk or as single species using normal phase, reverse phase and HILIC separation methods. In the reverse phase methods described herein, the labeled targets are separated by hydrophobic attraction to the separation substrate (matrix), C2-C18. The more hydrophobic labeled targets are more retained and elute with increasing organic content of the elution solution. The free unreacted label is more polar and elutes first and with appropriate choice of starting conditions; the free label and smaller aldehydes pass freely by the separation matrix. For HILIC separations, the mechanism of attraction is reversed with the more hydrophobic labeled targets eluting early and the less hydrophobic, smaller aldehyde, and free dye retained longer. In some embodiments, careful selection and matching of the labeling agent, target, separation matrix and separation conditions (solvent, pH, buffer (ion-pairing agent)) can be useful.

Provided herein are systems for detecting the presence of at least one carbonyl containing moiety in a sample. The systems comprise: a substrate to capture the carbonyl containing moiety; reagents for eluting the carbonyl containing moiety off the substrate; reagents for associating the carbonyl containing moiety with a reactive labeling agent; a column for resolving the labeled carbonyl containing moiety; solvents for eluting the labeled carbonyl containing moiety from the column; and a light and detector for generating fluorescence excitation, absorbance, and/or emission to detect the labeled carbonyl containing moiety. In some aspects, the system completes one cycle in less than about 2 hours. In some embodiments, the system further comprises standards for measuring the concentration of the at least one carbonyl containing moiety.

### Reactive Labeling Agents

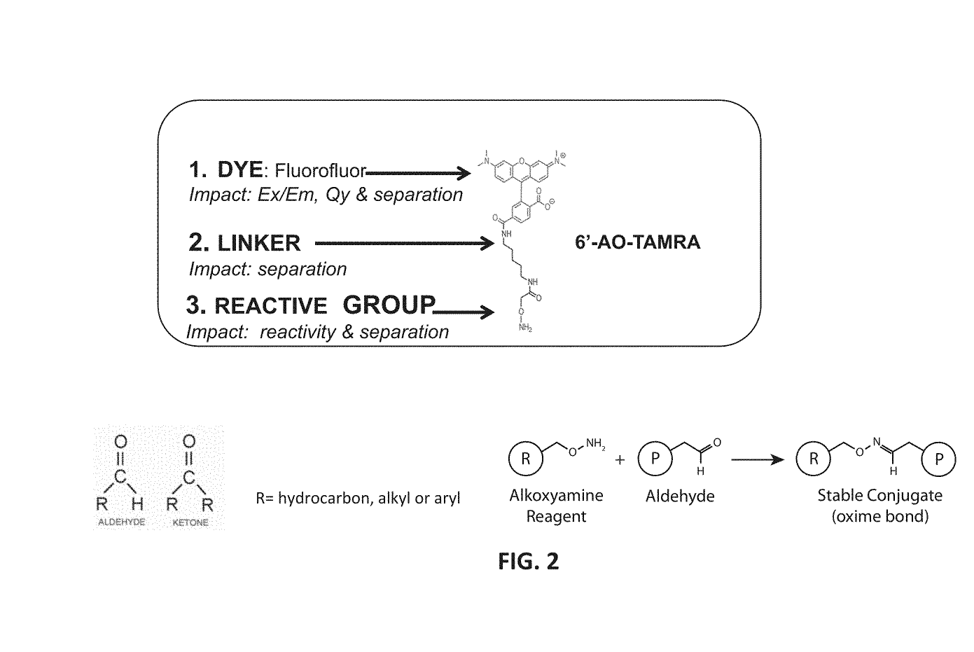

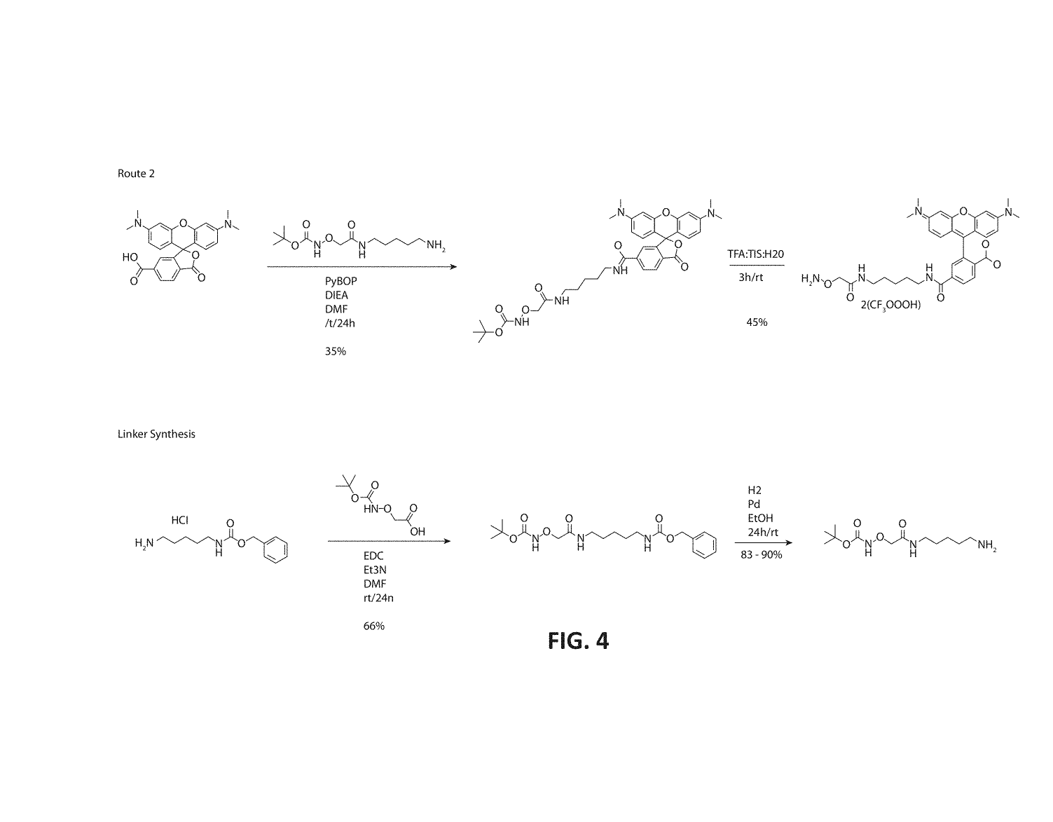

Exemplary reactive labeling agents were constructed to provide both selective and rapid labeling as well as single carbon separation (FIG. 2). One illustrative reactive labeling agent comprising ao-6-TAMRA and cadavarine provides rapid and selective coupling to carbonyl groups with aldehyde>>ketone reactivity (FIGS. 2, 3 and 4). The resulting oxime bond is more stable than complementary hydrozone bonds formed with hydrazine and hydrazide chemistry which require reduction to secondary amine linkage increased stability. Hydrozones are subject to scrambling due to re-equilibration.

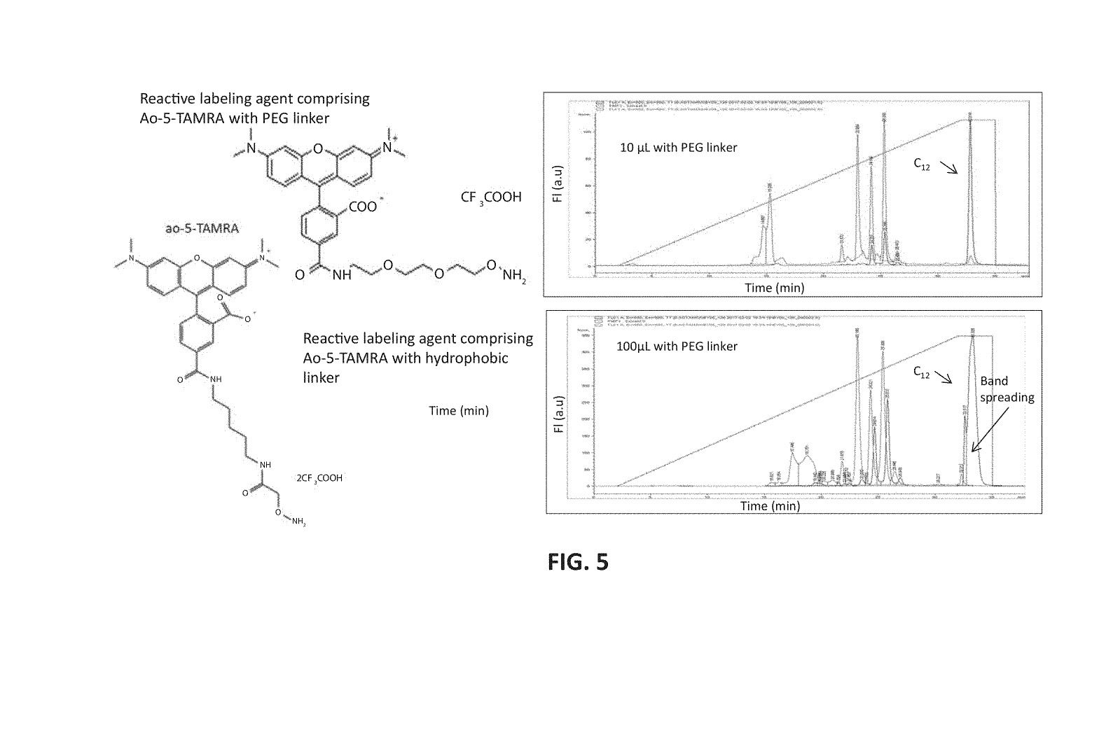

The reactive labeling agent contains three aspects which are varied for a given application. The parent fluorophore, for example, TAMRA, defines the detection modality and primary separation mechanism. The linker modulates the separation mechanism and quantum yield. For example substitution of the diamine alkyl linker for a more polar water soluble polyethylene (PEG) linker results less retention on reverse phase hydrophobic separation. The PEG linker restricts the volume that can be loaded due to band broadening as a result of lower affinity for the separation matrix compared to the alkyl diamine linker (FIG. 5). The last element, the reactive group modulates specificity, rate and label stability.

Typically, a reactive labeling agent can selectively and efficiently (rapidly) label the target carbonyls, can provide for bulk and individual separation from the unreacted reagent, and can provide adequate detection properties for spectroscopic detection.

Three structural aspects, described above, of the reactive labeling agent can be varied to provide options for labeling when varying the solvents, reaction times and temperatures, and column length.

The fluorophore can affect the detection and separation of the target carbonyls.

The linker can affect separation mechanism and quantum yield.

The reactive group can affect specificity, reaction rate, and label stability.

Thus, in some embodiments, the reactive labeling agent comprises a fluorophore, a linker, and a reactive group.

In some embodiments, the fluorophore is tetramethyl rhodamine (TAMRA), rhodamine X (ROX), rhodamine 6G (R6G), or rhodamine 110 (R110). In some embodiments, the fluorophore is aminooxy 5(6) TAMRA, or aminooxy 5 TAMRA, or aminooxy 6 TAMRA. In some embodiments, the fluorophore is a fluorescent hydrazine or aminooxy compound.

In some embodiments, the labeling reaction is selective for carbonyl functional groups: aldehydes and ketones with reactivity much greater for aldehydes than ketones (aldehyde>>than ketone). The reaction forms a stable oxime bond. Hydrazine and hydrazide reactive groups also provide selective labeling of carbonyls.

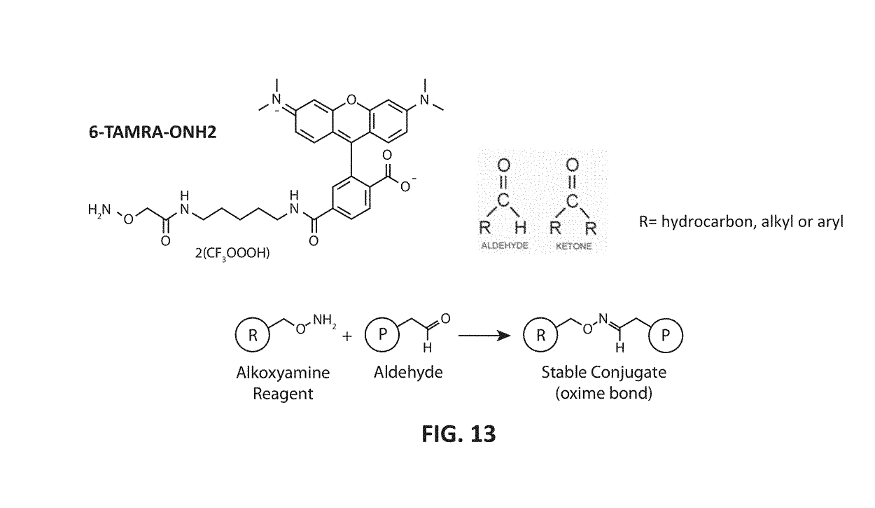

The nature of the fluorophore, TAMRA isomer, linker, and reactive group can modulate the reactivity as well as separation properties of the reactive labeling agent. However, other aspects of the reaction and separation processes can be modulated to achieve desirable reaction rates and efficiencies, including, for example, buffer (pH), catalyst, fluorophore concentration, or organic solvent. See FIG. 13.

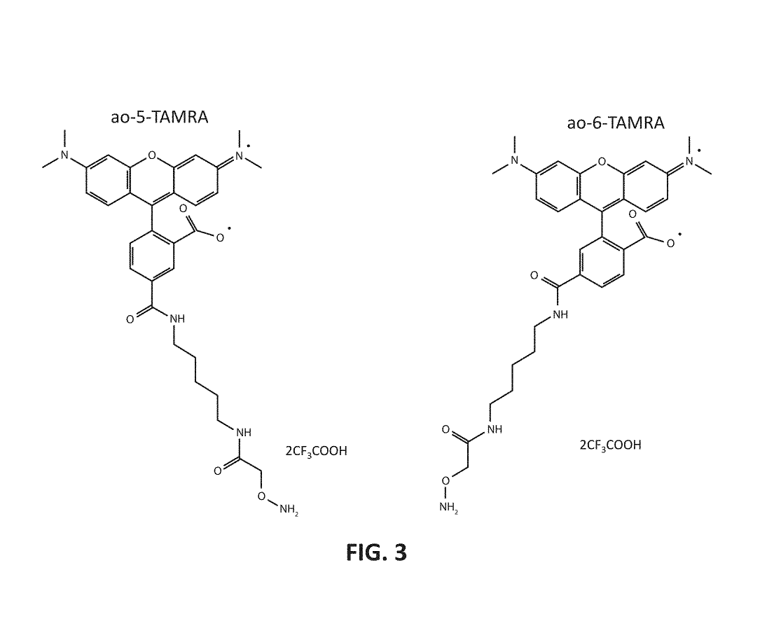

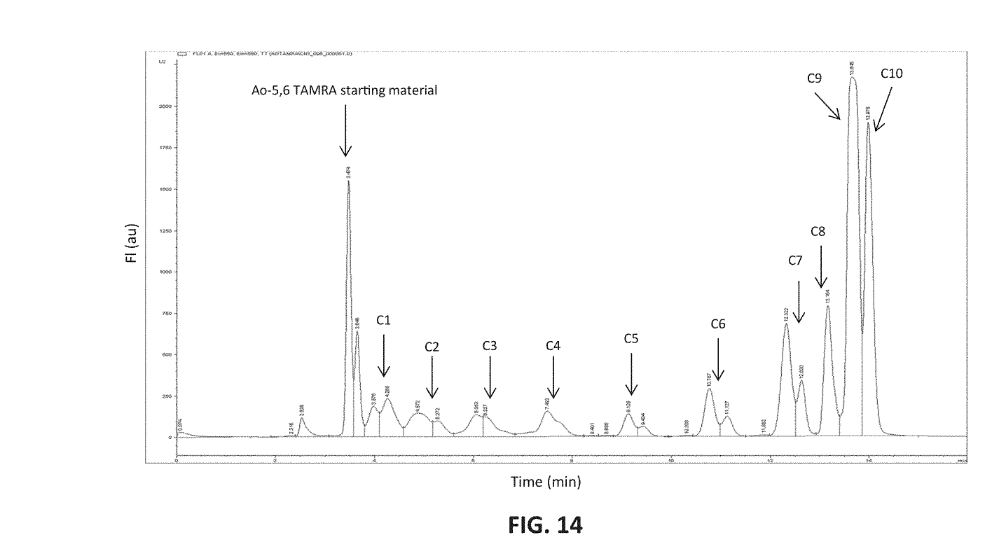

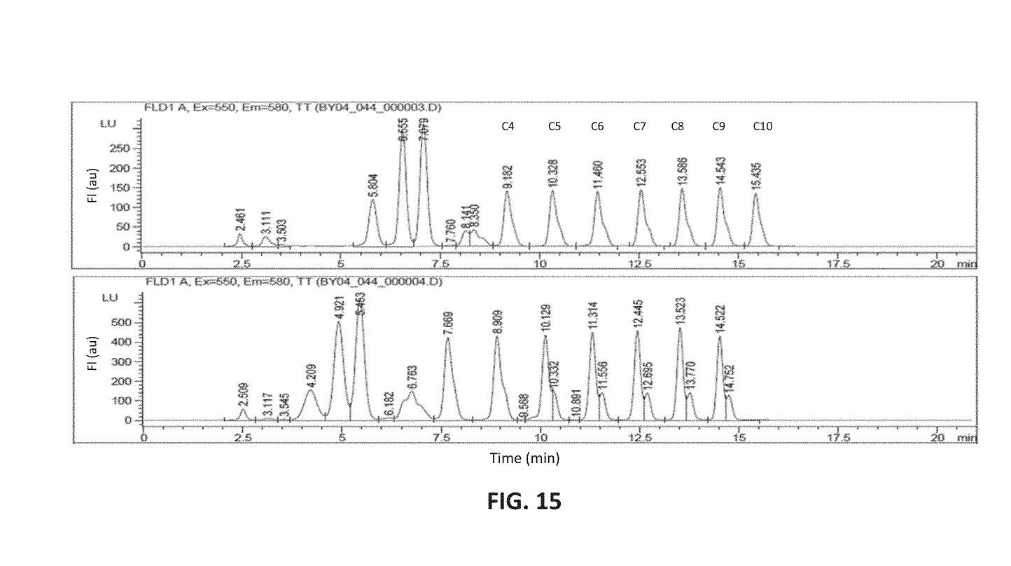

The reactive labeling agent can comprise a mixture of ao-TAMRA isomers modified according to the description provided herein: for example, ao-5-TAMRA and ao-6-TAMRA. See FIG. 3 for exemplary reactive labeling agents using both isomers. This mixture can vary in isomer ratio depending upon the synthesis and purification methods used. Use of the mixed isomer formulation yields a complex chromatograph: two bands for each aldehyde, one for each isomer. Resolution between individual aldehydes can be more difficult due to isomer overlap, though modification of the solvent system or column characteristics can reduce isomer separation but permit aldehyde resolution. See FIG. 14. Use of a single isomer formulation yields a less complex chromatograph than the mixed isomer formulation. The reactive labeling agent comprising the ao-6-TAMRA isomer is less retained in this method and allows for a shorter run time (less than 15 minutes) and better resolution of longer chain aldehydes than does the reactive labeling agent comprising the ao-5-TAMRA isomer (more than 15 minutes). See FIG. 15.

Reactive labeling agents comprising aminooxy-5(6)-TAMRA can react with aldehydes or ketones to form a stable oxime compound under mild conditions. See FIGS. 2 and 13.

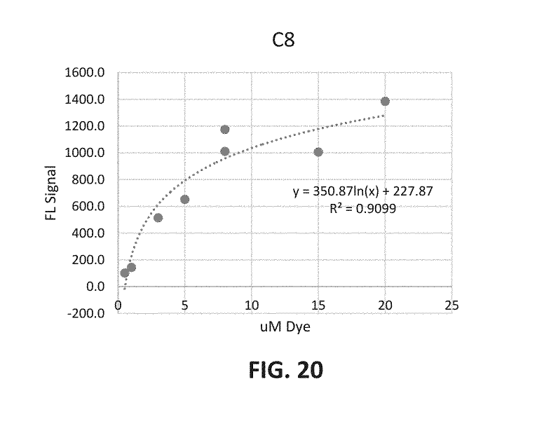

The concentration of the reactive labeling agent can be varied to achieve a desired fluorescence. In one experiment, the reactive labeling agent concentration varied from 0.5 μM to 20 μM, and maximum signal was observed at approximately 10 μM. See FIG. 20.

### Linkers and Reactive Groups

As mentioned previously, a linker can affect separation mechanism and quantum yield. For example, substitution of a diamine alkyl linker for a more polar water soluble polyethylene glycol (PEG) linker can result less in retention on reverse phase hydrophobic separation. Illustratively, a reactive labeling agent comprising ao-PEG-5-TAMRA is less retained on reverse phase chromatography than the corresponding reactive labeling agent comprising ao-TAMRA with a hydrophobic linker: 6 min versus 11 min (40% MeOH initial), respectively.

Even though adequate separation can be achieved using a 5% to 100% methanol gradient, the PEG linker restricts the volume that can be loaded onto a reverse phase column due to band broadening as a result of lower affinity for the separation matrix compared to a alkyl diamine linker. Appreciable band spreading is observed when the injection volume is increased from 10 μL to 100 μL. See FIG. 5.

Reactive labeling agents comprising ao-6-TAMRA can be present in injection volumes from 10 to 900 μM and still provide suitable separation and minimal to no band broadening. See FIG. 5.

Exemplary linkers include substituted alkyl-diamines (C2-C10), substituted amino-carboxylic acids (C2-C10), and substituted polyethylene glycols (N=1-10). In some embodiments, the linker is selected from the group consisting of hexanoic acid, aminohexanoic acid, cadavarine, polyethylene glycol, and polyglycol.

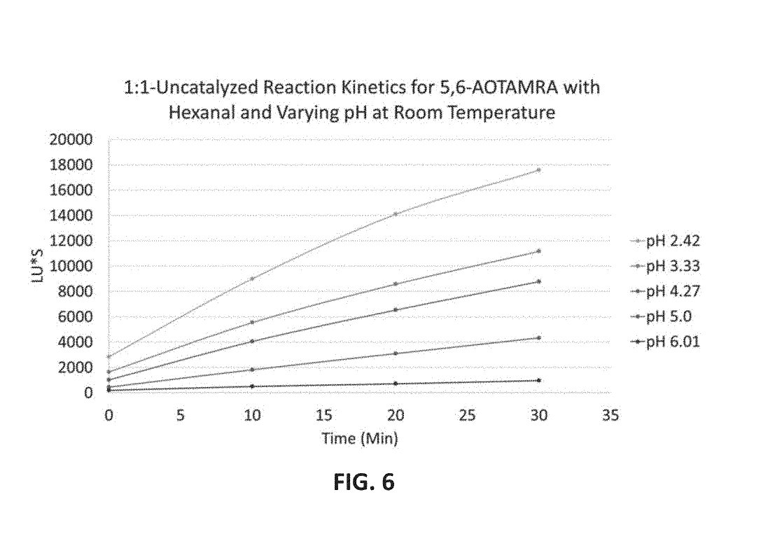

The reactive group provides specificity, rate of reaction, and label stability. For example, an aminoxy reactive group provides rapid formation of a stable oxime bond with carbonyl function groups. The reaction at ambient room temperature exhibits >90% conversion in 60 minutes in contrast to hydrazide couplings which can take several hours to overnight for similar conversion. The initial rate can be accelerated at elevated temperatures (2× at 40° C.). The reaction exhibits a pH profile with increasing reaction rate between pH 5 and pH 2.4. See FIG. 6. The rate at pH 4.2 is approximately 10× of the rate at pH 7.

In some embodiments, the reactive group can be selected from the group consisting of a hydrazine moiety, a carbohydrazide moiety, a hydroxylamine moiety, a semi-carbazide moiety, an aminooxy moiety, and a hydrazide moiety.

### Compounds

Provided herein are compounds comprising a fluorophore, a linker, and a reactive group. In some embodiments, the fluorophore is TAMRA, is aminooxy-5-TAMRA, is aminooxy-6-TAMRA, or is a mixture of aminooxy-5-TAMRA and aminooxy-6-TAMRA. In some embodiments, the linker is selected from the group consisting of hexanoic acid, aminohexanoic acid, cadavarine, polyethylene glycol, and polyglycol. In some embodiments, the reactive group is selected from the group consisting of a hydrazine moiety, a carbohydrazide moiety, a hydroxylamine moiety, a semi-carbazide moiety, an aminooxy moiety, and a hydrazide moiety.

In some embodiments, the compound is selected from the group consisting of:

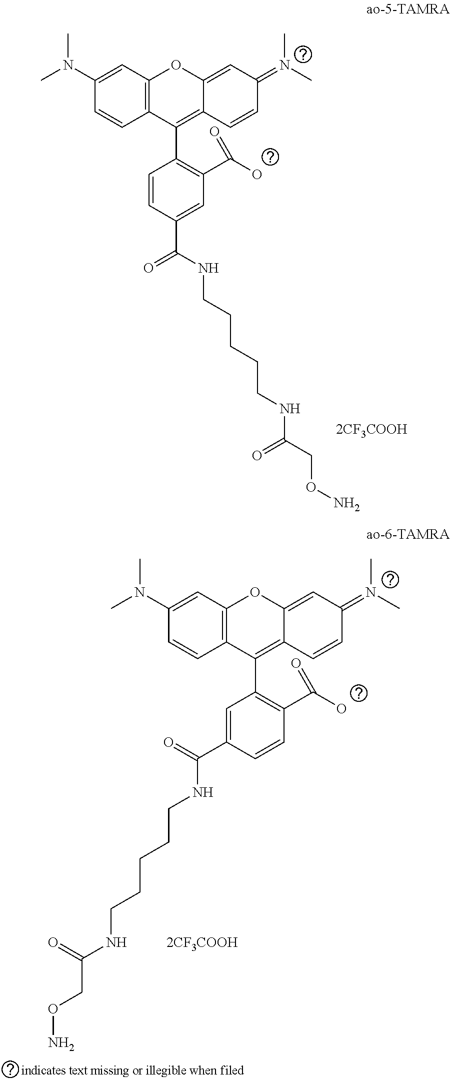

and mixtures thereof.

### Catalysts and Other Reaction Conditions

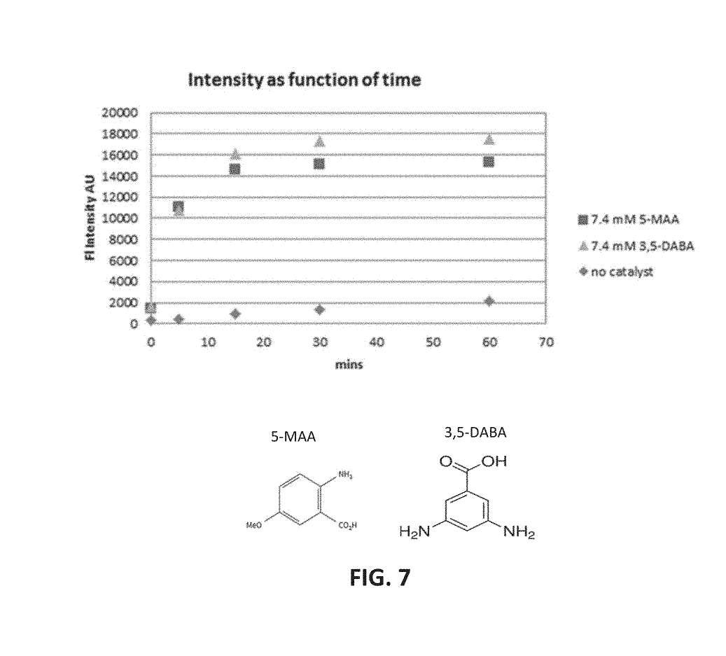

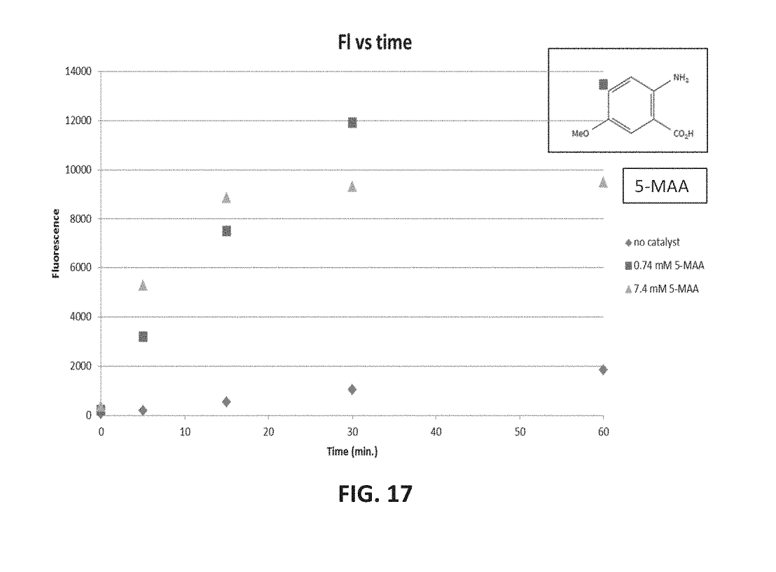

The reaction rate can be further enhanced by the addition of aromatic amine compounds such as 3,5 diamine benzoic acid (3,5 DABA) and 5-methoxy anthranilic acid (2-amino-5-methoxy-benzoic acid) (5-MAA). See FIG. 7. The reaction rate increased more than 10 times over the reaction without catalyst. 3,5-DABA has limited solubility at the desired pH and undergoes fairly rapid oxidation under the conditions employed, but can be utilized in appropriate situations. Use of the catalyst, 5-MAA in conjugation with acidic pH (30 to 70 mM citrate pH 4.2) yielded rapid coupling of the aldehyde to the reactive labeling agent comprising ao-6-TAMRA. See FIG. 7. A little as 1 pmole of aldehyde can be labeled in 15 mins at ambient temperature under these conditions. See FIG. 17. Additional catalysts are contemplated herein, including those described by Crisalli and Kool, each of which is incorporated by reference herein: Crisalli and Kool, Organic Letters 2013, 15(7): 1646-1649; Crisalli and Kool, Journal of Organic Chemistry 2013, 78: 1184-1189; Kool et al., Journal of American Chemical Society 2013, 135: 17663-17666.

In some embodiments, capture and labeling can be accelerated by the presence of catalysts such as 5-methoxyanthanlic acid (5-MAA), 3,5 diamino-benzoic acid (3,5-DABA) or similar catalysts, temperature and pH. In some embodiments, the pH is between 2 and 5, or less than about 5.

FIG. 7 provides an example of the impact of two different catalysts on the reaction rate for the standard solution method. As can be seen, a labeling reaction is extremely slow without a catalyst at low analyte concentrations. With a catalyst, the reaction rate can be much faster, for example, about 10 times faster. The reaction provides for a ratio of about 1:1.2 5,6 ao-TAMRA:hexanal as a function of 5-MAA (5-methoxy anthranilic acid or 2-amino-5-methoxy-benzoic acid) or 3,5 DABA (3,5-diaminobenzoic acid) in a molar ratio of about 1:900-1000 hexanal:catalyst and a ratio of about 1:1200, dye:catalyst. Conditions: 6.2 μM 5,6-ao-TAMRA, 7.5 μM hexanal. No buffer was added as the pH was buffered by the catalyst. See FIG. 7.

FIG. 17 provides an additional example of the impact of the catalyst 5-MAA, where the reactive labeling agent comprising 5,6 ao-TAMRA is present in a 1:1.2 ratio to hexanal as function of 5-MAA, at molar ratios of: 0, 100, and 1000. The concentration of the reactive labeling agent comprising 5,6-ao-TAMRA was 6.2 μM and the concentration of hexanal was 7.5 μM. The 6.5 mM citrate buffer had a pH 4.16, and the experiment was performed at room temperature. See FIG. 17.

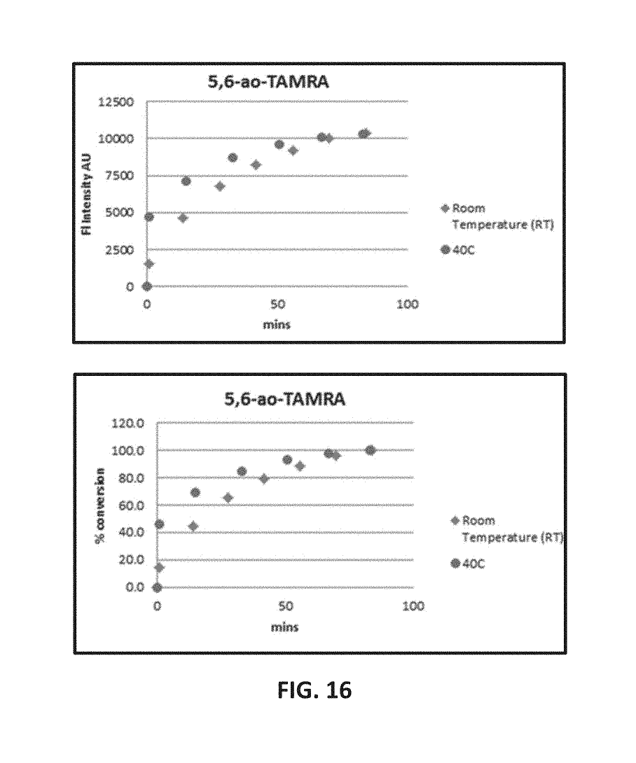

In a further example, the effect of temperature on the reaction rate was examined. As can be seen in FIG. 16, the increase in temperature primarily increased the initial rate of reaction. Experimental conditions were 1:1 ratio reactive labeling agent to hexanal, e.g. 7 μM a0-TAMRA with 7 μM hexanal, 30% ethanol, 75 mM citrate at pH 4.2. See FIG. 16.

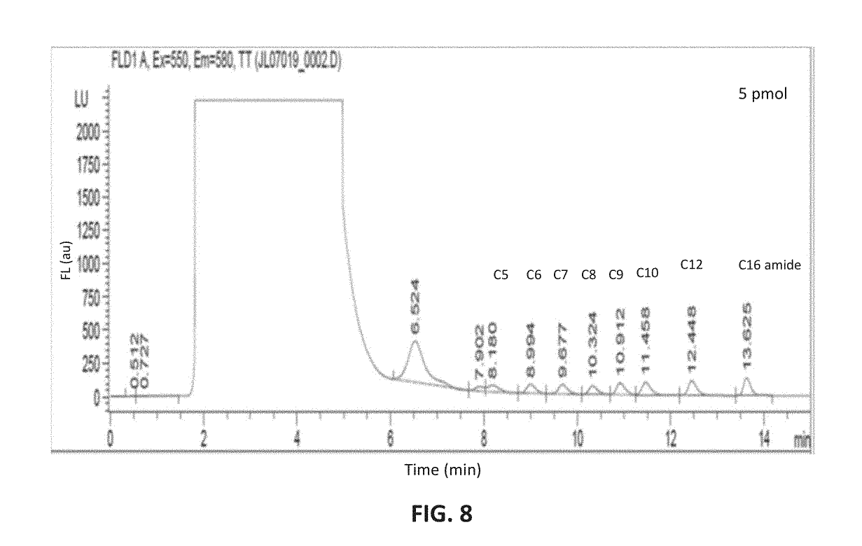

### Standards (See FIG. 8)

In some embodiments, standards are included in the assay. Standards can ensure consistency and can provide assurance that a given assay is functional and providing accurate data. In some embodiments, at least one reactive standard is included. In some embodiments, at least one non-reactive standard is included.

Internal standards should not interfere chromatographically with target molecule.

A reactive standard can provide a mechanism for correcting signals for drift in reactivity that could be caused by a number of factors including: reagent degradation (fluorophore, catalyst, buffer), dispensing variations, and environmental variations (temperature). Long chain aliphatic aldehydes can be selected and screened for the reactive standard.

A non-reactive standard can provide for normalization of signals due to instrument drift or variance, a measure of overall reactivity, and retention time registration. In some embodiments, a non-reactive standard is stable under the conditions employed, ie. does not undergo reactive or passive exchange with the reagents (i.e. labeling reagent, target, catalyst.) The non-reactive standard must be stable spectroscopically and chemically under the conditions of the assay. This requires special consideration in the selection and construction of a non-reactive standard. For the non-reactive standards, amide functionalized 6-TAMRAs can be prepared. Illustrative compounds include 6-TAMRA-C14, 6-TAMRA-C16, and 6-TAMRA-C18.

In some embodiments, a reactive or non-reactive standard compound does not interfere with the target compounds, for example, with C4-C10 aldehydes. In some embodiments, the reactive or non-reactive compounds are well resolved from one another. In some embodiments, the standard reactive standard compound has suitable reactivity for the assay. In some embodiments, the non-reactive linkage is stable to the reaction conditions.

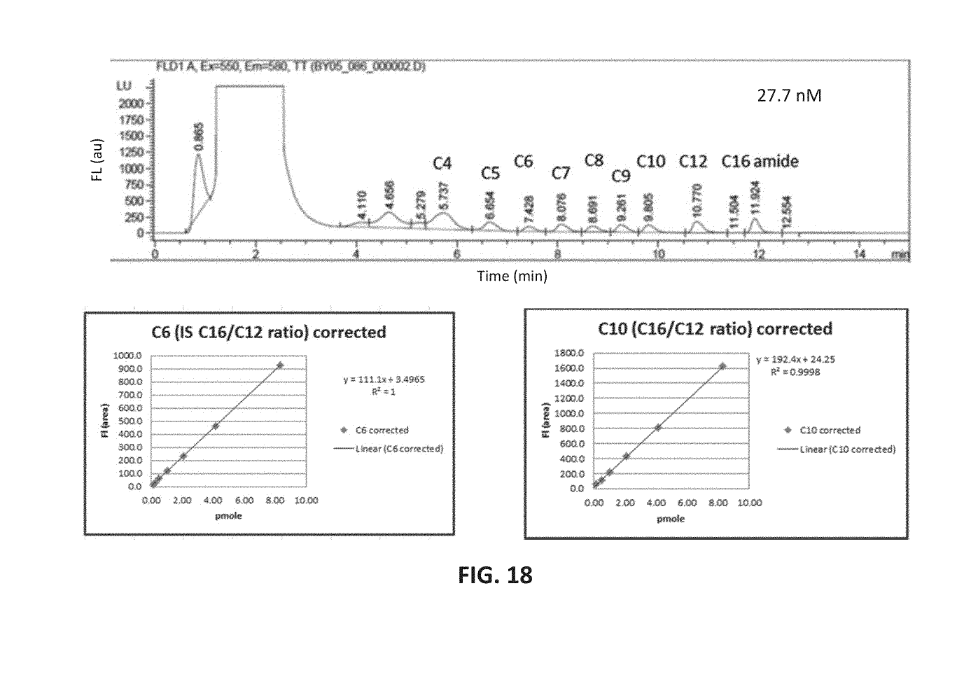

Using standards, the limit of detection (LOD) for a given method can be determined. In FIG. 18, a LOD curve was constructed using a serial dilution of a mixture of aldehydes in equal concentration. Reactive (C12 aldehyde) and non-reactive (C16 amide) internal standards were added at a constant concentration to each sample in the dilution series.

The reaction was incubated for 15 mins then quenched using 1 M sodium bicarbonate at pH 10. Mixtures were analyzed by HPLC using standard conditions, including 4×20 mm reverse phase C18 column (5 μm). In this example, the LOD was <0.13 pmole.

### Description of the Process

The method and strategy disclosed herein is illustrated in FIG. 1. Provided herein are methods and systems having both selective and specific reactivity in labeling the target, and specific rapid isolation and detection of the desired labeled targets. The target molecules, aldehydes and ketones for example, are labeled with carbonyl selective reactive fluorescent “paint” (See FIG. 1). The label can serve one or more of the following functions: transforms the optically “transparent” alkyl aldehyde targets into a species that can be observed and quantized by either absorption or fluorescence detection; and enables and enhances the selective isolation of the desired targets. The reactive label and separation matrix can provide the correct combination of reactivity, signal, and separation properties. In some embodiments, the methods provide the ability to resolve and identify individual aldehydes that differ by 1 carbon in chain length.

Aldehydes are deposited on silica, and can be washed off with methanol in 30 mM citrate buffer at pH 4.2. A double internal standard can optionally be added, as can a reactive aldehyde mimic, a catalyst, and the reactive labeling agent. The mixture is incubated, for an amount of time sufficient for the labeling reaction to occur. The reaction can be quenched with a basic solution, for example, sodium bicarbonate, etc.

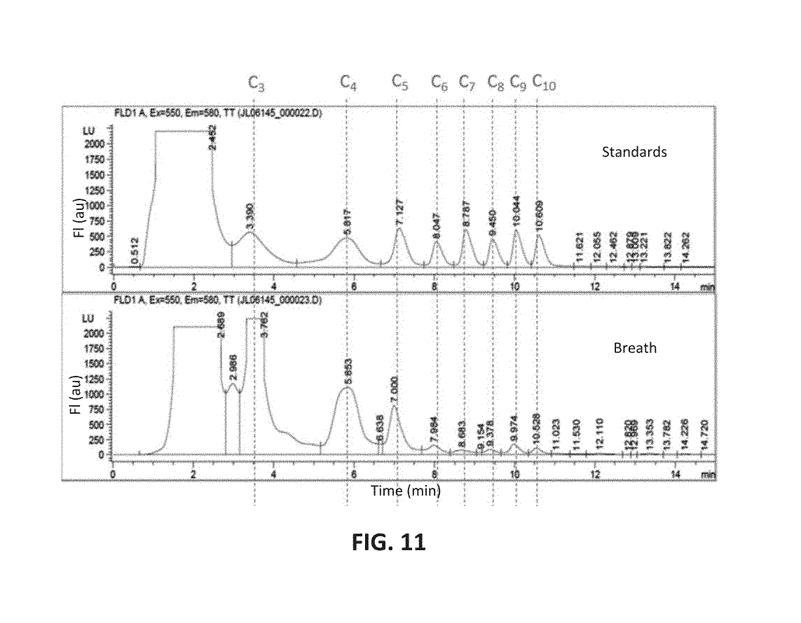

The solution is then injected into C18 reverse phase separation column which has been pre-equilibrated with a low to moderate organic content solvent/buffer mixture such as 45% MeOH/TEA pH 7. Following injection, the sample is subject to gradient of increasing organic solvent content. The gradient can be linear, stepwise or a combination (step+linear). A typical gradient process can be initial pre-equilibration 45% MeOH/TEA pH 7; followed by hold 2-4 mins; followed by linear increase over 10 mins from 45%/MeOH pH 7 to 100% MeOH; followed by rapid return to the initial conditions (45% MeOH/TEA pH 7). During this process labeled aldehydes (labeled target) elute from the column based on the combined hydrophobicity of the target/label. For those labeled with ao-6-TAMRA, the elution order is from smaller chain aldehydes to larger chain aldehydes (C3, C4, C5 . . . C10). The description here is illustrative though other solvents and other solvent gradients are contemplated herein. Using the elution solution containing a TAMRA derivative as an illustrative example, the labeled CCM is eluted and detected by measuring the fluorescence absorbed or emitted by the TAMRA derivative attached to the CCM. See FIG. 11.

The aldehyde content is quantitated by monitoring the signal for each eluting species. The signal is a function of the initial aldehyde concentration. With a continuous flow detection with is synchronized with the elution gradient the signal is monitored as a function of time following injection. The signal intensity and area reflects the population of each labeled species (labeled aldehyde). Quantitation for each species in a sample is by reference to a standard curve generated by injection of known quantities of synthesized labeled-aldehyde standards. Aldehydes can also be quantitated using a dis-continuous flow detection where labeled species are step-wise eluted and the fluorescence signal measured for each group using standard fluorimeter or similar device. The quantitation process described is an example of “end-point” assay scheme. In this scheme the assay is allowed to incubate for a set time and then analyzed. The conversion or signal increase is a function of the initial carbonyl (target) concentration. There are two general assay format or detection modes. They are generically described as end-point and kinetic. In an end-point assay the system is incubated for a set time and the signal is read. The signal at that point reflects the amount of analyte in the system. For a positive assay, the greater the concentration of the analyte, the greater the signal increase. In a kinetic assay the rate of change is monitored for a set duration. The rate of change is correlated to the amount of analyte. In some aspects, the end-point assay is employed with the methods provided herein.

In yet another embodiment, a two-solution methodology is used. After the substrate is loaded with the CCM, the CCM is eluted into a first elution solution or “rinse” solution comprising generally 30% ethanol, 50 mM citrate, and 30% ethanol at pH 2.5. The Agent is added to the rinse solution thereby resulting in painted CCM. This solution is then passed through another substrate, for example, a silica frit stack, to capture the painted CCM. The painted CCM is then eluted from the substrate with the painted CCM captured therein using a second elution solution or “rinse” solution comprising greater than 50% acetonitrile and 90% ethanol. In this embodiment, target CCMs are grouped into classes. The number of classes depends on the number of different rinses used. In an SPE type of format one, two or three rinses are used to separate short chain (C1-C3), medium chain (C4-C7) and long chain (C8-C10) labeled aldehydes. The groups can be quantitated based on fluorescence signal using either a continuous or discontinuous flow method as describe above. One of the benefits of this second embodiment is that it provides a rapid assessment of total aldehydes and target groupings of aldehydes. This can facilitate rapid screening processes.

In some aspects, the systems and methods permit a user the ability to resolve and identify individual aldehydes that differ by one carbon in chain length. Illustratively, within the system, the components of the breath sample are eluted with a first elution solution to form a carbonyl containing moieties solution. The carbonyl containing moieties solution is then mixed with a reactive labeling agent to form a solution that includes painted carbonyl containing moieties therein. The painted carbonyl containing moieties are then captured on the separation filter assembly or second filter assembly. The painted carbonyl containing moieties are then eluted by gradient to allow resolution and detection of carbonyl containing moieties differing by a single carbon in chain length.

The desired painted carbonyl containing moieties can be isolated and separated from unreacted label and interfering species using reverse phase (RP), normal phase (NP), ion exchange (IC), and or hydrophilic (HILIC) chromatography. The desired species can be isolated individually for analysis and quantitation or as groups of species. For example, using moderate size C18 matrices (nominal 40-60 μm particles), C4-C10 linear alkyl carbonyls can be isolated form the unreacted label and smaller linear alkyl carbonyls (C1-C3) using a two-step elution process, for example, 40% MeOH followed by a 90% MeOH elution. In this example, desired species are group analyzed as a sum of species. Individual alkyl aldehydes can be isolated and analyzed using smaller bead size C18 matrices (10 μm) using a linear, step, or piece wise (step followed by linear) gradient. For example, in embodiment provided herein, individually labeled carbonyl moieties are isolated and analyzed employing reverse phase separation using a column containing 10 μm C18 particles using a 45% to 90% MeOH piece wise gradient at moderate pressures (≈700 psi) (See FIG. 19).

### Detection

Painted carbonyl species are detected, analyzed, and quantitated by direct light, within a predetermined wavelength range through the solution, thereby producing fluorescence. The fluorescence is detected, analyzed and quantitated within a predetermined wavelength range. For example, when using aminooxy-5(6)-TAMRA, the λ_{Ex}/λ_{Em}(in MeOH) is 540/565 nm; when using aminooxy-5(6)-ROX, the λ_{Ex}/λ_{Em} (MeOH) is 568/595 nm.

Analysis can be performed in a static mode (bulk quantitation) or in a flowing mode (individual analysis) as a function of time as the solution is eluted from the separation matrix and passes the detector window, or via a hybrid flow and stop mode.

In some embodiments, the step of detecting the CCM comprises measuring fluorescence emission produced by excitation of the fluorophore. In some embodiments, the step of detecting the CCM comprises measuring fluorescence absorbance produced by excitation of the fluorophore. In some aspects, the step of detecting the CCM comprises directing light within a predetermined wavelength range to the labeled CCM, thereby producing a fluorescence, and detecting the fluorescence. In some aspects, the concentration of the CCM is determined by calculating the fluorescence absorption or emission relative to a standard curve, wherein the fluorescence signal is proportional to the concentration of the CCM.

The system is also very amenable to use with a “stop” solution. Elevation of the pH to more than 9 by the addition of sodium bicarbonate or sodium hydroxide quenches the reaction providing the ability to batch process samples for delayed analysis.

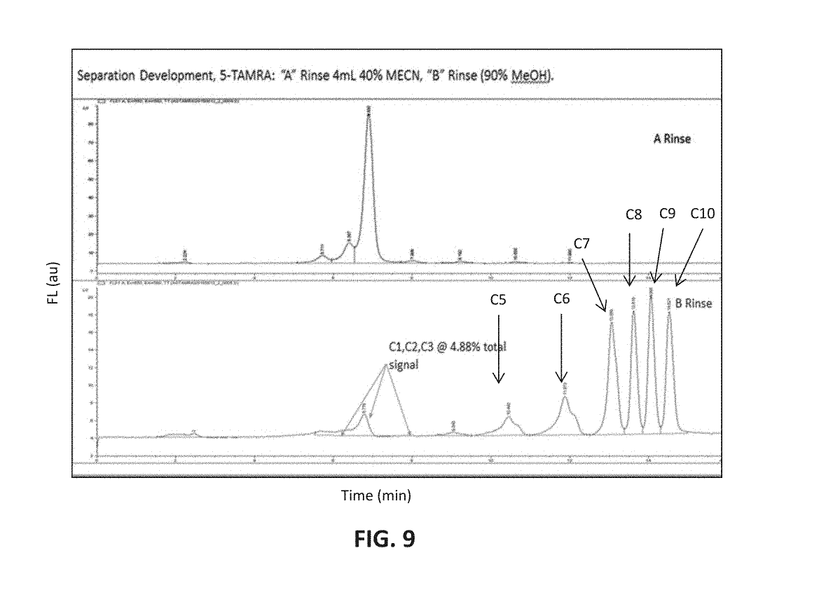

As described the reactive label and corresponding labeled aldehydes can be isolated and separated using a manual SPE format process or by rapid chromatography using semi-prep or analytical short columns. In the SPE format illustrated in FIG. 9, the labeled aldehyde targets are loaded onto a standard conditioned SPE column. Two rinses are employed. The initial rinse releases unreacted label, C1, C2 and C3 labeled aldehydes into one fraction. A final rinse of high organic content results in release of longer chain aldehydes. These include C5-C10. The carryover is <4% in this example. The C5-C10 can be quantitated optically (absorbance or fluorescence) to provide a sum of aldehydes in the sample. The grouping can be modulated by varying the formulation of the rinses.

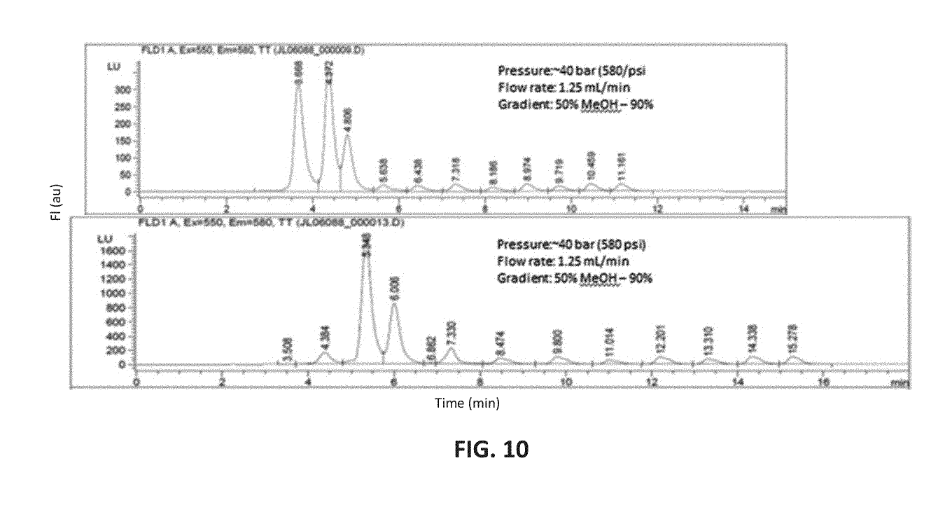

A more surprising attribute, is the ability to rapidly isolate and quantitate trace levels of aldehydes which differ by signal carbon chain lengths using semi-prep chromatography medium 10-15 μm particle C18. Single carbon resolution and detection is illustrated using a 4.6×30 mm and 4.6×50 mm column containing 10 μm materials as moderate pressures in less than 15 minutes. See FIG. 10.

The method provides for rapid detection and quantitation of trace levels of alkyl aldehydes. Sub-picomoles of aldehydes can be quantitated following 15 minutes of incubation and separation, with a total time approximately 35 minutes. Employing a reactive and nonreactive internal standard pair for correction of reaction efficiency an LOD of <0.13 pico mole can be observed. See FIG. 18.

Optically, labeled aldehydes can be detected down to 1 to 10 femto moles depending upon the sensitivity of the detector. See FIG. 8. Very trace levels of aldehydes can be detected by extending the incubation time and increasing the column length to provide for additional resolution.

A reactive labeling agent comprising ao-6-TAMRA in combination with a buffer and catalyst can detect and quantitate aldehydes in breath samples. (See FIGS. 12 and 13). In the examples provided fluorescence emission detection is employed. Aldehyde labeling and identification was confirmed by LCMS analysis (data not shown). As a corollary, the labeling scheme is amenable to dual Fl/LCMS detection or single Fl and mass spec detection modalities.

Furthermore the methods and systems provided herein are amenable to both biological and environmental samples for trace aldehyde targets of interest. The disclosure is not limited to solution or gas (air) based sampling but can be adapted to other samples for use of real time application or point of care applications and provide data within 2 hours post sampling.

Unless the context clearly requires otherwise, throughout the description and the claims, the words “comprise,” “comprising,” and the like are to be construed in an inclusive sense, as opposed to an exclusive or exhaustive sense; that is to say, in the sense of “including, but not limited to.” Where the context permits, words in the above Detailed Description using the singular or plural number may also include the plural or singular number respectively. The word “or” in reference to a list of two or more items, covers all of the following interpretations of the word: any of the items in the list, all of the items in the list, and any combination of the items in the list.

The above-detailed description of embodiments of the disclosure is not intended to be exhaustive or to limit the teachings to the precise form disclosed above. While specific embodiments of and examples for the disclosure are described above for illustrative purposes, various equivalent modifications are possible within the scope of the disclosure, as those skilled in the relevant art will recognize. Further, any specific numbers noted herein are only examples: alternative implementations may employ differing values, measurements or ranges. It will be appreciated that any dimensions given herein are only exemplary and that none of the dimensions or descriptions are limiting on the present disclosure.

The teachings of the disclosure provided herein can be applied to other systems, not necessarily the system described above. The elements and acts of the various embodiments described above can be combined to provide further embodiments.

Any patents and applications and other references noted above, including any that may be listed in accompanying filing papers, are incorporated herein by reference in their entirety. Aspects of the disclosure can be modified, if necessary, to employ the systems, functions, and concepts of the various references described above to provide yet further embodiments of the disclosure.

These and other changes can be made to the disclosure in light of the above description. While the above description describes certain embodiments of the disclosure, and describes the best mode contemplated, no matter how detailed the above appears in text, the teachings can be practiced in many ways. Details of the system may vary considerably in its implementation details, while still being encompassed by the subject matter disclosed herein. As noted above, particular terminology used when describing certain features or aspects of the disclosure should not be taken to imply that the terminology is being redefined herein to be restricted to any specific characteristics, features or aspects of the disclosure with which that terminology is associated. In general, the terms used in the following claims should not be construed to limit the disclosures to the specific embodiments disclosed in the specification unless the above Detailed Description section explicitly defines such terms. Accordingly, the actual scope of the disclosure encompasses not only the disclosed embodiments, but also all equivalent ways of practicing or implementing the disclosure under the claims.

Accordingly, although exemplary embodiments have been shown and described, it is to be understood that all the terms used herein are descriptive rather than limiting, and that many changes, modifications, and substitutions may be made by one having ordinary skill in the art without departing from the spirit and scope of the invention.

What is claimed is:

1. A method for detecting the presence of at least one aldehyde in a sample, the method comprising the steps of:
exposing the sample to a substrate to capture the aldehyde;
eluting the aldehyde off the substrate;
mixing the aldehyde with a reactive labeling agent;
injecting the labeled aldehyde onto a column;
eluting the labeled aldehyde from the column in an organic solvent; and
detecting the labeled aldehyde;
wherein the method of detecting is complete in less than about 2 hours.

2. The method of claim 1, further comprising measuring the concentration of the at least one aldehyde.

3. The method of claim 1, wherein the at least one aldehyde is selected from the group consisting of a C1 aldehyde, a C2 aldehyde, C3 aldehyde, a C4 aldehyde, a C5 aldehyde, a C6 aldehyde, a C7 aldehyde, a C8 aldehyde, a C9 aldehyde, a C10 aldehyde, and mixtures thereof.

4. The method of claim 1, wherein the at least one aldehyde is aliphatic, a di-aldehyde, or an aromatic aldehyde, or mixtures thereof.

5. The method of claim 1, wherein the sample comprises two or more aldehydes of different carbon chain lengths, and wherein the step of detecting the labeled aldehyde resolves each aldehyde.

6. The method of claim 1, wherein the sample is a biological sample.

7. The method of claim 1, wherein the sample is an environmental sample.

8. The method of claim 1, wherein the sample is selected from the group consisting of a breath sample, a urine sample, a blood sample, a plasma sample, and a sample of the headspace in a culture.

9. The method of claim 1, wherein the sample is a breath sample.

10. The method of claim 1, wherein the substrate is selected from the group consisting of silica, polysaccharides, cellulose acetate, nitrocellulose, polyether sulfone, polyethylene, nylon, polyvinylidene fluoride (PVDF), polyester, polypropylene, silica, deactivated alumina, diatomaceous earth, MgSO₄, porous matrix, vinyl chloride, vinyl chloride-propylene copolymer, vinyl chloride-vinyl acetate copolymer, cloth, cotton, nylon, rayon, porous gels, silica gel, agarose, dextran, gelatin, polymeric film, and polyacrylamide.

11. The method of claim 1, wherein the step of capturing the aldehyde comprises collecting at least one aldehyde on a filter assembly.

12. The method of claim 1, wherein the reactive labeling agent comprises a fluorophore, a linker, and a reactive group.

13. The method of claim 12, wherein the fluorophore is selected from the group consisting of tetramethyl rhodamine (TAMRA), aminooxy 5(6) TAMRA, aminooxy 5 TAMRA, aminooxy 6 TAMRA, rhodamine X (ROX), rhodamine 6G (R6G), rhodamine 110 (R110), and a coumarin.

14. The method of claim 12, wherein the linker is selected from the group consisting of substituted alkyl-diamines (C2-C10), substituted amino-carboxylic acids (C2-C10), and substituted polyethylene glycols (N=1-10).

15. The method of claim 12, wherein the linker is selected from the group consisting of hexanoic acid, aminohexanoic acid, cadavarine, polyethylene glycol, and polyglycol.

16. The method of claim 12, wherein the reactive group is selected from the group consisting of a hydrazine moiety, a carbohydrazide moiety, a hydroxylamine moiety, a semi-carbazide moiety, an aminooxy moiety, and a hydrazide moiety.

17. The method of claim 1, wherein the reactive labeling agent is selected from the group consisting of 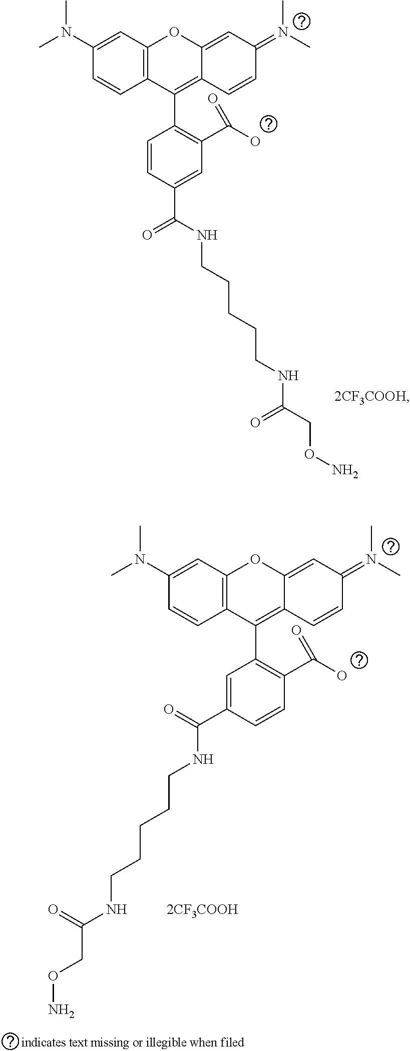
and mixtures thereof.

18. The method of claim 1, wherein the step of detecting the aldehyde comprises measuring fluorescence emission produced by excitation of the fluorophore.

19. The method of claim 1, wherein the step of detecting the aldehyde comprises measuring fluorescence absorbance produced by excitation of the fluorophore.

20. The method of claim 1, wherein the step of detecting the aldehyde comprises directing light within a predetermined wavelength range to the labeled aldehyde, thereby producing a fluorescence, and detecting the fluorescence.

21. The method of claim 2, wherein the concentration of the aldehyde is determined by calculating the fluorescence absorption or emission relative to a standard curve, wherein the fluorescence signal is proportional to the concentration of the aldehyde.

22. The method of claim 1, wherein the column is a reverse phase column.

23. The method of claim 1, wherein the organic solvent is selected from the group consisting of methanol, isopropanol, acetonitrile, and ethanol.

24. A method for detecting the presence of at least one carbonyl containing moiety in a sample, the method comprising the steps of:
exposing the sample to a substrate to capture the carbonyl containing moiety,
eluting the carbonyl containing moiety off the substrate,
mixing the carbonyl containing moiety with a reactive labeling agent,
injecting the labeled carbonyl containing moiety onto a column,
eluting the labeled carbonyl containing moiety from the column in an organic solvent, and
detecting the labeled carbonyl containing moiety,
wherein the method of detecting is complete in less than about 2 hours.

25. The method of claim 24, wherein the carbonyl containing moiety is selected from the group consisting of aldehydes, ketones, carboxylic acids and mixtures thereof.

26. A method of detecting carbonyl containing moieties in a gas sample, the method comprising:
isolating carbonyl containing moieties from a sample;
mixing the carbonyl containing moieties with a reactive labeling agent, wherein the carbonyl containing moieties associate with the reactive labeling agent;
passing the labeled carbonyl containing moieties through a column;
exciting the labeled carbonyl containing moieties exiting the column; and
detecting the carbonyl containing moieties by measuring the fluorescence emitted from or absorbed by the reactive labeling agent associated with the carbonyl containing moieties,
wherein the step of detecting resolves the carbonyl containing moieties based on the carbon chain length, and
wherein the time elapsed from isolating the carbonyl containing moieties from the sample to detecting the carbonyl containing moieties is less than about 2 hours.

27. A compound comprising a fluorophore, a linker, and a reactive group.

28. The compound of claim 27, wherein the fluorophore is selected from the group consisting of ao-5-TAMRA, ao-6-TAMRA, and mixtures thereof.

29. The compound of claim 27, wherein the linker is selected from the group consisting of hexanoic acid, aminohexanoic acid, cadavarine, polyethylene glycol, and polyglycol.

30. The compound of claim 27, wherein the reactive group is selected from the group consisting of a hydrazine moiety, a carbohydrazide moiety, a hydroxylamine moiety, a semi-carbazide moiety, an aminooxy moiety, and a hydrazide moiety.

31. The compound of claim 27, comprising:

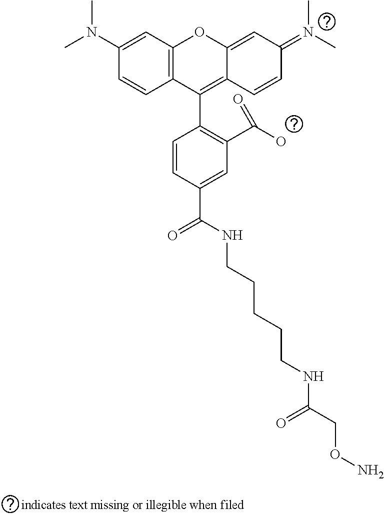

32. The compound of claim 27, comprising:

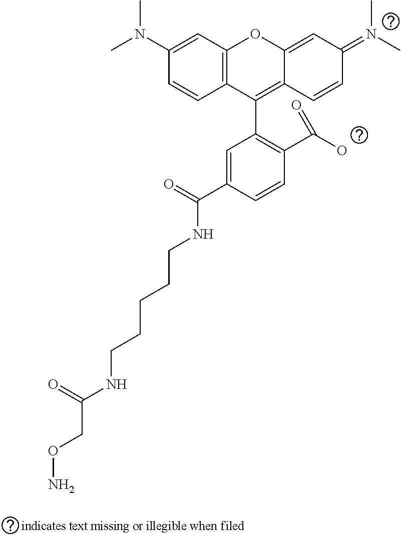

33. A system for detecting the presence of at least one carbonyl containing moiety in a sample, the system comprising:
a substrate to capture the carbonyl containing moiety;
one or more reagents for eluting the carbonyl containing moiety off the substrate;
one or more reagents for associating the carbonyl containing moiety with a reactive labeling agent;
a column for resolving the labeled carbonyl containing moiety;
one or more solvents for eluting the labeled carbonyl containing moiety from the column; and
a light and detector for generating fluorescence excitation, absorbance, and/or emission to detect the labeled carbonyl containing moiety.

34. The system of claim 33, wherein the system completes one cycle in less than about 2 hours.

35. The system of claim 33, further comprising one or more standards for measuring the concentration of the at least one carbonyl containing moiety.
